(19) United States
(12) Patent Application Publication
(10) Pub. No.: US 2008/0214680 A1
(43) Pub. Date: Sep. 4, 2008

(54) Application of arsenic as a cancer prevention agent

(76) Inventor: Gary Michael Kayajanian, Lake Worth, FL (US)

Correspondence Address: GARY KAYAJANIAN, 514 Holyoke Lane, Lake Worth, FL, 33467, US

(21) Appl. No.: 11/788,306

(22) Filed: Apr. 19, 2007

Related U.S. Application Data
(63) Continuation-in-part of application No. 10/634,869, filed on Aug. 6, 2003, now abandoned.

(51) Int. Cl. A61K 47/02; A61P 35/00

(52) U.S. Cl. 514/769

(57) ABSTRACT

Arsenic in drinking water at levels between 25 and <75 μg/L is cancer protective, compared to levels between 0 and <25 μg/L and levels at or above 75 μg/L. Methods are presented to decrease the cancer rate or not increase the cancer rate, for specific cancers and for cancers in general, based on this observation.

This application is a continuation-in-part of application Ser. No. 10/634,869.

### CROSS REFERENCE

Bae, I.-J. U.S. Pat. No. 6,309,672 (Oct. 30, 2001).

### NON PATENT LITERATURE DOCUMENTS

Kayajanian, G. “Arsenic, cancer and thoughtless policy” (2003). Ecotoxicology & Environmental Safety 55: 139-142.

Table [10-] A at pages 308-309 NAS/NRC Report on Arsenic in Drinking Water (1999). National Academy Press.

Morales, K H, Ryan L, et al. “Risk of Internal Cancers from Arsenic in Drinking Water (2000). Environmental Health Perspectives 108(7): 655-661.

EPA Repot (2000). “A Re-Analysis of Arsenic-Related Bladder and Lung Cancer Mortality in Millard County, Utah. [Five pages, plus two pages of graphs and a 53 page data set.]

Cuzick, J, Evans, S et al. (1982). “Medicinal Arsenic and Internal Malignancies.” British Journal Cancer 45: 904-910.

Copies of these documents were provided in the parent application; they will be provided again by request of the patent examiner

### FEDERALLY FUNDED RESEARCH AND DEVELOPMENT STATEMENT

This invention was not supported by Federal funds.

### BACKGROUND OF THE INVENTION

This invention relates to the extended use of low exposures of arsenic in water-soluble form as a cancer-prevention agent in humans. The body of scientific data supporting the cancer claim consists of three epidemiology data sets. The American data set also supports a heart disease claim in men, though that claim is not made as part of this application.

In common lore, arsenic, including KH₂AsO₄ and As₂O₃, is viewed as an acute poison when used at high exposures. Forms of arsenic have found many uses as a killing agent (e.g., insecticide, fungicide, and pesticide), even though arsenicals have been represented with more gentle language, like wood preservative. Arsenic has been the subject of regulatory review as a harmful contaminant in drinking water. At the high exposures found in the Himalaya and Andean groundwater, arsenic has been characterized as carcinogenic at multiple tissue sites. In 1976 under the Safe Drinking Water Act, the U.S. Environmental Protection Agency (EPA) proposed an interim maximum contaminant level of 50 micrograms per liter (μg(L) as part of the National Interim Primary Drinking Water Standards. NRC (National Research Council) (1999). Arsenic in Drinking Water. (This inventor assumes all American water systems are compliant with the 50 μg/L standard, delivering water at or below 50 μL arsenic.) More recently, EPA Administrator Whitman affirmed Administrator Browner's purposeful but scientifically oblivious decision to lower that arsenic contaminant standard to 10 μg/L, relying on a strict monotonic extrapolation to zero exposure of a high dose bladder cancer mortality measure excess in the Taiwan data set.

The NRC Report cited above documents arsenic's high dose uses as treatment for a broad spectrum of symptoms and illnesses, especially in the later half of the nineteenth century; it also comments on the lack of data supporting the claim of “essentiality” for arsenic, even though it is found universally in living organisms.

A low dose cancer prevention claim for arsenic parallels an earlier claim made for dioxin by this inventor, but is clearly not derivative: dioxin is a specific cyclic organic chemical which binds preferentially to a pair of cell-produced agents; the resulting complex passes into the nucleus and binds to DNA, where it may act as a promoter blocker of cancers. The subject of this invention is the broad collection of arsenic molecules, for which no known binding system or any possible cancer prevention mechanism has been identified. NRC (1999). There is no published evidence that arsenic binds to the Ah receptor and/or the Arnt protein, which are the components of the dioxin binding system. Kayajanian U.S. Pat No. 6,444,698. The beneficial effects (prevention of cancers) attributed to arsenic in this invention is tied to a modest level of exposure to arsenicals (between 25 and <75 μg/L) in drinking water over an extended period of time. Lower and higher levels of arsenic exposure are comparatively and significantly harmful. Kayajanian, Ecotox. and Environ. Safety 55, 139-142 (2003). Any claims made by others for human treatments involving high dose arsenic exposures, as described below, are for cancer treatment, not prevention.

Bae U.S. Pat No. 6,309,672 claims that arsenic hexoxide (As₄O₆), a polycyclic compound isolated and purified from a natural product or reproduced in the laboratory, at pharmaceutical doses is a treatment for malignant cancers sensitive to it—specifically human cancers of the uterus, lung, maxillary sinus, kidney and urinary bladder. In his human cancer treatment, the effective daily dose of arsenic hexoxide was 4 grams (for an average treatment of two years), approximately 40,000 times greater than the arsenic exposure of 50 ppb (the equivalent of 50 μg/L) in drinking water called for in this invention: 4 grams daily for a Bae patient compared to 100 μg in two liters of water daily for an American. A significantly effective cancer prevention treatment in women and men, which Bae does not claim or demonstrate in humans, at less than 0.01% his arsenic hexoxide dosage for the arsenic compounds found in drinking water is sufficiently novel to justify a patent for this invention on the cancer claims. Bae explains his high dose experimental observations as apoptosis, a form of cell destruction.

Bae cites a paper by Chen et at. (May 1, 1997) in his references, published more than a year before his patent filing, which recites the use of arsenic trioxide (As₂O₃) as an acute treatment on acute prontyelocytic leukemia cells. The treatment is effective over an exposure range of 0.5-2.0 μmoles per liter, but ineffective at 0.1 μmoles per liter. These values are equivalent to 0.1-0.4 μl/ml and 0.02 μg/ml, respectively. Chen requires just 9-36 percent of Bae's arsenic hexoxide dose to produce an acute effect on cells with arsenic trioxide. (Two percent of Bae's dose [0.1 μmoles per liter] is an in eve cellular dose for arsenic trioxide.) For treating humans, Bae's prolonged daily exposure to arsenic hexoxide is 1818-times greater than his effective cellular dose and 40,000-times greater than 50 ppb. Chen reports no human exposure data, but if he treated cancers in humans with an 1818-times greater daily dose of arsenic trioxide, that exposure would be 3600-14,400-times greater than this invention's 50 ppb or μg/L. Chen's ineffective dose would be 720-times greater than 50 μg/L.

Abstracts in Bae's references list arsenic trioxide or other arsenicals, usually in conjunction with other compounds, as apopotosic agents. What distinguishes the present invention from Chen et al. and Bae's other references is the inherent safety attached to lifetime exposures of a 50 μg/L level of arsenic in drinking water. After all, if arsenicals were as apoptosic by themselves as those papers and abstracts claim at the 50 μg/L in drinking water, humans should not escape the first twenty years of life, when cell growth primarily occurs, without enormous, even greater levels of cell death. These other inventions rely on arsenicals as “-cide” agents, killing growing cells preferentially.

### SUMMARY OF THE INVENTION

Arsenic is an elemental chemical. It and most of its salts are r soluble in water, many inorganic arsenic salts are natural components of drinking water. The measure of arsenic in drinking water is usual presented as parts per million (ppm), parts per billion (ppb) or micrograms per liter (μg/L). EPA regulations for arsenic have employed the μg/L measure, which has ppb as a numeric equivalent. This invention identifies levels of arsenic between 25 and <75 μg/L in drinking water as preventative of several kinds of cancers compare to lower and higher levels. The invention does not make the claim that this level of arsenic cures existing cancers. EPA has long claimed that arsenic is carcinogenic at high levels of exposure at multiple tissue sites and that lowering the arsenic level in drinking water from levels above 1,000 μ/L will reduce cancer mortality rates—the greater the reduction in arsenic level, EPA claims, the greater the cancer reduction. What is novel about this inventor's claim is that reducing the arsenic level in drinking water below 25 μg/L will significantly increase the cancer mortality, not only in the cancer classification EPA chose to base its regulation on but on others. The inventor relies on three epidemiology data sets, including the Taiwan bladder cancer mortality data EPA relied on to justify lowering the arsenic in drinking water standard to 10 μg/L. [EPA would have lowered the standard to 3 μg/L, slightly above the detect level for arsenic, if the regulatory process the Agency followed allowed it to ignore the additional increased costs for arsenic removal associated with 3 μg/L compared to 10 μg/L.] Why should the Inventor's analysis of the Taiwan bladder cancer mortality data be preferred to the EPA's analysis? The EPA Administrator asked the regulatory question, “Should the upper bound level of arsenic in drinking water be kept at 50 μg/L or should it be lowered?” The Administrator selected bladder cancer mortality as the health endpoint and the Taiwan epidemiology study set as the data source for the regulatory decision. The Inventor compared the bladder cancer mortality rate in the five villages with “around 50 μg/L arsenic in drinking water”[actually, between 42 and 60 μg/L] and compared it to the bladder cancer mortality rate in the five villages with lower arsenic levels [between 10 and 32 μL]. In men, the bladder cancer mortality rate [0.465 bladder cancer deaths per thousand person-years (tp-y)] associated with the arsenic levels between 10 and 32 μg/L is more than three-and-a-half times higher than the bladder cancer mortality rate [0.120 bladder cancer deaths per tp-y] in the villages with arsenic drinking water levels between 42 and 60 μg/L. In women, the bladder cancer mortality rate [0.648 bladder cancer deaths per tp-y] associated with the arsenic levels between 10 and 32 μg/L is more than four times higher than the bladder cancer mortality rate [0.161 bladder cancer deaths per tp-y] associated with arsenic drinking water levels between 42 and 60 μg/L. In each sex, the bladder cancer death rate increases associated with the arsenic level between 10 and 32 μg/L compared to between 42 and 60 μg/L is significant. EPA regulators missed the finding because they modeled the data from the 42 villages in the study, first broad grouping the villages into four exposure categories [((0 to 100), (>100 to 300), (>300 to 600) and (>600 μg/L)], then extrapolated linearly the bladder cancer mortality rate from the highest to the lowest of the four arsenic exposure category. Extrapolation is a useful modeling technique, when applied correctly. The purpose of modeling is to explain data. But when, as here, the conclusions of the model are contradicted by real data, those conclusions must be ignored as scientifically unsound.

This pattern of the bladder cancer mortality findings developed by the Inventor is repeated in the liver and lung cancers in the same data set. Total cancer findings, especially in women, in the Millard County, Utah data set (collected and published on by EPA scientists, but ignored by EPA regulators) and by the Cuzick et al. study of British medical patients dosed with Fowler's solution support and allow the expansion of the Inventor's claims. Specifically, in the Millard County data set, the total cancer death rate is 9.189 cancers per 100 women with arsenic in drinking water exposures between 0 and <25 μg/L compared to 2.784 cancers per 100 women with arsenic in drinking water exposures been 25 and <75 μg/L—a hugely significant difference (p<0.000001). The USEPA regulatory claims on arsenic were generated from the evaluation of a single cancer endpoint in a single epidemiology study. This Inventor's patent claims rest on the evaluation of three long-term epidemiology studies, including the study the Agency relied on for its regulatory decision. A more detailed discussion of these studies follows, below.

In the one American epidemiology data set examined for this invention, average daily arsenic intake ranged from 0−<175 ppb. In the Taiwan data set of 42 villages, also discussed below, villagers had calculated exposures ranging from 10 ppb to 934 ppb; one well had arsenic levels measured as high as 1,752 ppb. A number of published papers report on the level of total arsenic assayed in human blood, urine, hair and nails. NRC (1999).

The EPA regulators and the scientists (other than this inventor) who have examined high dose arsenic epidemiology studies have claimed arsenic is carcinogenic at multiple tissue sites, like lung, liver, bladder, kidney, colon and skin in both men and women. Chen, C. J., et al., Cancer Res. 45, 5895-5899 (1985). Also see, Morales, K. H., Ryan, L. M. et al., Environ. Health Perspect. 108, 655-661 (2000); Smith, A. H., et al., Science 296, 2145-2146 (2002); NRC (1999); and EPA, Report: Arsenic in Drinking Water Rule Economic Analysis (EPA 815-R-00-026) (2000). Underlying all the arsenic cancer characterization is the ingrained notion (supported over an exposure range above 25 μg/L) that reducing exposure to this chemical, designated a carcinogen at high exposure, will result in a reduction of the cancer risk over the fill range of exposures (i.e., from high exposure down to zero exposure. The inventor has reanalyzed available data from three epidemiology data sets, including the Taiwan data set EPA relied on to reduce the maximum allowable arsenic level in drinking water from 50 ppb to 10 ppb: all three demonstrate a J-shaped cancer mortality response to exposure—in the two data sets amenable to a detailed numeric analysis, the cancer mortality trough is associated with an arsenic in drinking water level around 50 ppb (42 to 60 ppb in one data set, 25 to <75 ppb in the other). Kayajanian, G., (2003).

The Taiwan data set, published at pp. 308-309 of the 1999 NRC Report, cited above, reports on lung, liver and bladder cancer mortality in men and women in 42 Taiwan villages. These deaths are associated with population sizes presented as man- or woman-years. The arsenic exposure for all villagers in a village is determined by a direct current assay of the arsenic level in the well or wells supplying each village. (When more than one water well serves a village, the arsenic level for each well is rank ordered with that of the others, and the arsenic level of the middle well is taken by the NRC to represent exposure; when an even number of wells supply a village, the average of the middle two wells measures the arsenic exposure of those villagers.) The cancer measure used for the men and women in each village is lung, liver, and/or bladder cancer deaths per thousand person years. For the five villages reporting arsenic levels near 50 ppb (42-60 ppb), the former regulatory standard, the cancer measures are 0.53 for men and 0.51 for women for the pooled three cancer classifications. For the five villages with lower arsenic levels (10-32 ppb), the cancer measures are significantly greater (p<0.001 for each sex): 1.65 for men and 1.62 for women. Of course, the cancer measure gradually increases for villagers with arsenic levels above 60 ppb—to a peak of 2.43 for men and 1.99 for women at an arsenic exposure range of 650-698 ppb, thus accounting for the J-shape. A similar J-shaped curve exists for the three individual cancer classifications for each sex.

The American data set was collected and published on by EPA scientists, who reported on sources of mortality in Millard County, Utah and the rest of the state. Lewis, D. R., et al., Environ. Health Perspect. 107, 359-365(1999). Lewis et al. compared mortality in Millard County with that in the rest of the state. This inventor sought to avoid the issues of confounding inherent in the Lewis et al. paper, choosing to compare residents of Millard County with each other, grouping within each sex by mean daily arsenic exposure in water from unpublished Agency data, provided to him by request. Total cancer mortality is the endpoint and total cancer mortality per 100 people is the cancer measure. The cancer measure for men is about 10% lower (7.220 versus 8.047, not significant) for men with a mean arsenic exposure between 25 and <75 ppb compared to men between 0 and <25 ppb. The cancer measure is 69% lower (2.784 versus 9.189, p<0.000001) for women with a mean exposure between 25 and <75 ppb compared to women between 0 and <25 ppb. At the next higher arsenic exposure range (between 75 and <125 pp)), the total cancer measure increases both for men (11.355, p<0.08) and women (9.504, p<0.01), thus accounting for the J-shape asserted above. Chen et al. (1985) associated high dose exposures of arsenic in men and women with significant increases in lung, liver, bladder, kidney, and skin and colon cancer mortality. When the sum of these six cancer classifications becomes the cancer measure for women, a greater (76%) but a less significant (p<0.01) reduction in mortality is associated with arsenic exposures between 25 and <75 ppb arsenic exposure in drinking water compared to exposures between 0 and <25. See, Kayajanian, G. (2003). The total cancer benefit in women associated with arsenic exposures from between 25 and <75 ppb occurs throughout life, but is most significantly evident from ages 60-79. Kayajanian, unpublished.

According to the NIH's SEER Cancer Statistics Review: 1973-1990 (1993), there are 570,000 cancers cases and 249,000 cancer deaths annually among women. If arsenic levels in drinking water are currently just barely in compliance with the old 50 ppb standard, lowering the arsenic level to 10 ppb should result in an annual increase of 1,087,000 cancers and 475,000 cancer deaths. This equates to 2979 extra cancers and 1301 extra cancer deaths per day, when the Millard County cancer data are applied nationally. If arsenic levels are compliant with the 10 ppb standard (that is <25 μg/L), raising that level to between 25 and <75 ppb should result in an annual decrease of 373,000 cancers and 163,000 cancer deaths. This equates to 1024 fewer cancers and 447 fewer cancer deaths per day, when the Millard county cancer data are applied nationally. As a practical matter, most water systems currently deliver water to most Americans with arsenic levels below 10 ppb. So the greater benefit of this invention to the population would appear as a cancer or cancer mortality reduction, rather than an avoidance of a cancer or cancer mortality increase.

The small data set, from the 1982 Cuzick et al. article in the British Journal Cancer (45, 904-911, especially Table 3) is structurally different from the Taiwan and Utah data sets. The pooled population of men and women were intentionally dosed with arsenic for medicinal purposes and standardized cancer mortality was presented as a function of two variables: increasing total dose and time in years since the first dose. Here, unlike Taiwan and Utah, the outside reference serves as the lowest exposure group and the lowest dosed group (<500 mg) serves as the next lowest expose group. Over all time intervals 10 cancers were observed in the lowest dosed group and 18.34 were expected from the outside reference (p<0.06). At the higher dosings, 14.41 cancers were expected, 24 were observed.

The Cuzick data also may be helpful in suggesting the effect of arsenic on cancer mortality. In the five years following the initial arsenic medication, 6.45 cancers deaths were expected; in the second five years, 6.67—a total of 13.12 cancer deaths. Over those ten years, 13 cancer deaths were recorded: but only 2 occurred in the first five years and 11 occurred in the second five years p<0.02)—a timing which suggests additionally that, irrespective of exposure level, arsenic delays cancer deaths.

A more detailed discussion of the Taiwan; Utah and Cuzick data sets are found in Kayajanian (2003), referenced above. That paper and other papers and reports cited in this patent are incorporated herein.

Methods for assaying the arsenic level in drinking water over the range of interest for this invention are evidenced in the Taiwan and Utah data sets. Arsenic's use as a medicinal (Fowler's (aqueous) solution in Cuzick et al.) attests to a method to purify and concentrate arsenic from one arsenic source to be added to waters with less than 25 μg/L arsenic (even waters with less than 50 μg/L arsenic) to raise the arsenic level of the treated water to between 25 and <75 μg/L. Alternatively, natural water sources with high arsenic levels could be mixed with waters with less than 25 μg/L arsenic to raise the level of the mixed drinking waters to between 25 to <75 μg/L.

### NOVELTY OF THE INVENTION

The Inventor's claim with respect to arsenic in drinking water is counter intuitive. Common sense and the USEPA's misread of the Taiwan data set would have the general public believe that the less arsenic in drinking water, the better: the more arsenic removed from drinking water, the better. In 2001, that Agency would have lowered the arsenic level to three (3) μg/L, had cost concerns made that requirement prohibitively expensive. The novelty of the Inventor's claim has three components: First, for drinking water sources with arsenic levels at or above 75 μg/L, the arsenic level should be reduced to between 25 and <75 μg/l, not lowered further: even though the Inventor and the USEPA each make the same observation that cancer mortality rates are reduced as arsenic levels are reduced from levels above 1000 μg/L to between 25 and <75 μg/L, the Inventor and the Agency fashion different cancer reduction remedies based on our different assessment of cancer data in water with arsenic levels below 25 μg/L. Second, for drinking water sources with arsenic levels between 25 and <75 μg/L, the arsenic level should be maintained, not lowered. Third, for drinking water sources with arsenic levels below 25 μg/L, the arsenic level should be increased to bring it between 25 and <75 μg/L, not decreased to 10 μg/L or maintained at or below 10 μg/L.

### IMPLEMENTATION OF THE INVENTION

Water is made available to the public for drinking in unspecified quantities through the tap, or by bottling specified smaller quantities. The water distributor has an opportunity, and in some cases the duty to test and publicly report the arsenic level in drinking water whether it will be delivered to the consumer through the tap or in bottles. (1) If the arsenic level in drinking water is between 25 and <75 μg/L, no effort need be made to raise or lower it. (2) If the arsenic level is below 25 μg/L, then 50 μg arsenic/L could automatically be added to the water intended for tap and/or bottle distribution before the distribution. Alternatively, a different amount of arsenic could be added to the to-be-distributed water to raise the arsenic level to fall between 25 and <75 μg/L. Further, individuals being supplied with drinking water by tap or bottle with arsenic levels below 25 μg/L could increase the arsenic levels by adding calibrated doses of arsenic to raise the arsenic level 25 or 50 μg/L for fixed amounts of water, like an eight ounce glass, a 16 ounce bottle, or a five gallon jug. Individuals without specific knowledge of the arsenic level in their drinking water could assume the level to be between zero and 10 μg/L, and add sufficient calibrated arsenic to increase the level to between 25 and 75 μg/L. (3) For water supplies with arsenic levels at or above 75 μg/L, several arsenic removal methods approved by the USEPA are available—generally, with greater cost attached to the lower level of arsenic remaining. None of these methods are practical for individuals to remove or lower arsenic levels from his/her glass, bottle or jug of water. The arsenic treatment processes also create waste products regarded by Federal and State Regulators as hazardous waste, the removal of which is costly and fraught with regulatory requirements. There is less waste product created in lowering high levels of arsenic in drinking water to just below 75 μg/L than to the Federal Standard of 10 μg/L. There is a greater opportunity to mix drinking water sources (blending some sources at or above 75 μg/L with others between 0 and 75 μg/L to generate a mix with less than 75 μg/L arsenic) to avoid altogether the creation of a hazardous waste product than there is under the current 10 μg/L arsenic regulatory standard (where some water sources would have arsenic levels at or above 75 μg/L, others at or below 10 μg/L and the mixed water needs to have arsenic at or below 10 μg/L). In foreign lands, the removal of arsenic from drinking water with levels even well above 1,000 μg/L need not be as stringent as the US standard of 10 μg/L to have the greater health benefit: removal to between 25 and <75 μg/L is most effective, and less costly.

Formulation of the arsenic added to drinking water to increase its level in drinking water to between 25 and <75 μg/L may take any of three forms: (1) the (aqueous) formulations of arsenic found natural at levels above 25 μg/L in some existing water supplies, that water suppliers would mix with water sources with arsenic levels below 25 μg/L; (2) those formulations, KH2AsO4 (aqueous or dried), or As₂O₃ (aqueous or dried) would be added to drinking water supplies by water suppliers to elevate the arsenic levels to between 25 and <75 μg/L; and (3) specific doses of those formulations, KH2ASO4 or As₂O₃that would add either 25 or 50 μg/L arsenic to specific drinking water quantities, such as eight or 16 ounces or a five gallon jug. [Fowler's Solution, the medicinal studied be Cuzick is a one percent aqueous solution of KH₂AsO₄.]

### DESCRIPTION OF THE PREFERRED EMBODIMENTS

There are a number of attributes associated with this invention:
(1) Daily arsenic exposure in drinking water between 25 and <75 μg/L is associated with a significant (p<0.000001) 69% reduction of total cancer deaths in women compared to women exposed to between 0 and <25 μg/L. (American data set.)
(2) Liver and bladder cancers are significantly reduced in men and in women in villages with 42-60 μg/L daily arsenic exposure compared to those in villages with 10-32 μg/L. (Taiwan data set.)
(3) Following full implementation, current regulatory policy, which compels water systems to lower or maintain arsenic levels at or below 10 μg/L, is expected to result in 1024 extra cancers diagnosed and 447 extra cancer deaths per day in women compared to full implementation of this patent invention.
(4) For some segments of the population, arsenic levels in water can be raised to between 25 and <75 μg/L by drinking water distributors or providers, by the addition of inorganic arsenic to drinking water, and/or mixing water from multiple sources.
(5) For some segments of the population with an arsenic level currently between 25 and <75 μg/L, not complying with the Federal 10 μg/L arsenic-in-drinking-water standard is the better alternative.
(6) For other segments of the population exposed to drinking water from the tap or bottled water with arsenic levels below 25 μg/L, individuals may choose to supplement arsenic in their diet by the addition of fixed amounts of arsenic (say, the equivalent of 25 or 50 μg/L) to fixed amounts of their drinking water.

What is claimed is:

1. A method for reducing total cancer morbidity and mortality in women by adjusting the arsenic level in drinking water to between 25 and <75 μg/L.

2. A method according to claim 1 when the cancer is lung cancer.

3. A method according to claim 1 when the cancer is liver cancer.

4. A method according to claim 1 when the cancer is bladder cancer.

5. A method according to claim 1 when the cancer is kidney cancer.

6. A method according to claim 1 when the cancer is skin cancer.

7. A method according to claim 1 when the cancer is colon cancer.

8. A method for reducing cancer mortality in men by adjusting the arsenic level in drinking water to between 42 and 60 μg/L.

9. A method according to claim 8 when the cancer is lung cancer.

10. A method according to claim 8 when the cancer is liver cancer.

11. A method according to claim 8 when the cancer is bladder cancer.

12. A method for not increasing total cancer morbidity and mortality in humans by maintaining the arsenic level in drinking water between 25 and <75 μg/L even though such maintenance would violate the newly enacted 10 μg/L upper bound EPA standard.

13. A method according to claim 12 when the cancer is lung cancer.

14. A method according to claim 12 when the cancer is liver cancer.

15. A method according to claim 12 when the cancer is bladder cancer.

16. A method according to claim 12 when the cancer is kidney cancer.

17. A method according to claim 12 when the cancer is skin cancer.

18. A method according to claim 12 when the cancer is colon cancer.
